(19) United States
(12) Patent Application Publication
(10) Pub. No.: US 2016/0331658 A1
(43) Pub. Date: Nov. 17, 2016

(54) HAIR CONDITIONING COMPOSITION COMPRISING A ZWITTERION OR PROTEINCAEOUS MATERIAL

(71) Applicants: Richard Mackenzie READ, Tarporley (GB); Hannah Mary SOUTHEY, Great Sutton (GB)

(72) Inventors: Richard Mackenzie READ, Tarporley (GB); Hannah Mary SOUTHEY, Great Sutton (GB)

(73) Assignee: Conopco, Inc., d/b/a UNILEVER, Englewood Cliffs, NJ (US)

(21) Appl. No.: 15/112,868

(22) PCT Filed: Jan. 22, 2015
(86) PCT No.: PCT/EP2015/051215

§ 371 Date: Jul. 20, 2016

(30) Foreign Application Priority Data
Jan. 23, 2014 (EP) 14152330.8

(51) Int. Cl. A61K 8/34; A61K 8/41; A61K 8/04; A61Q 5/12

(52) U.S. Cl. CPC A61K 8/342; A61Q 5/12; A61K 8/416; A61K 8/042; A61K 2800/49

(57) ABSTRACT

Conditioning composition comprising from 0.4 to 8% wt. fatty alcohol having from 8-22 carbons, from 0.1 to 2% wt. cationic surfactant component, water, and wherein the composition has a Draw Mass of from 1 to 250 g and comprising a zwitterion or proteinaceious material.

The present invention relates to a conditioning composition comprising superior conditioning capability.

Despite the prior art there remains a need for improved conditioning compositions.

Accordingly, the present invention provides a conditioning composition according to claim 1, 2, 3, 4 or 5.

The present invention will now be described, by way of example, with reference to the accompanying drawings, in which;

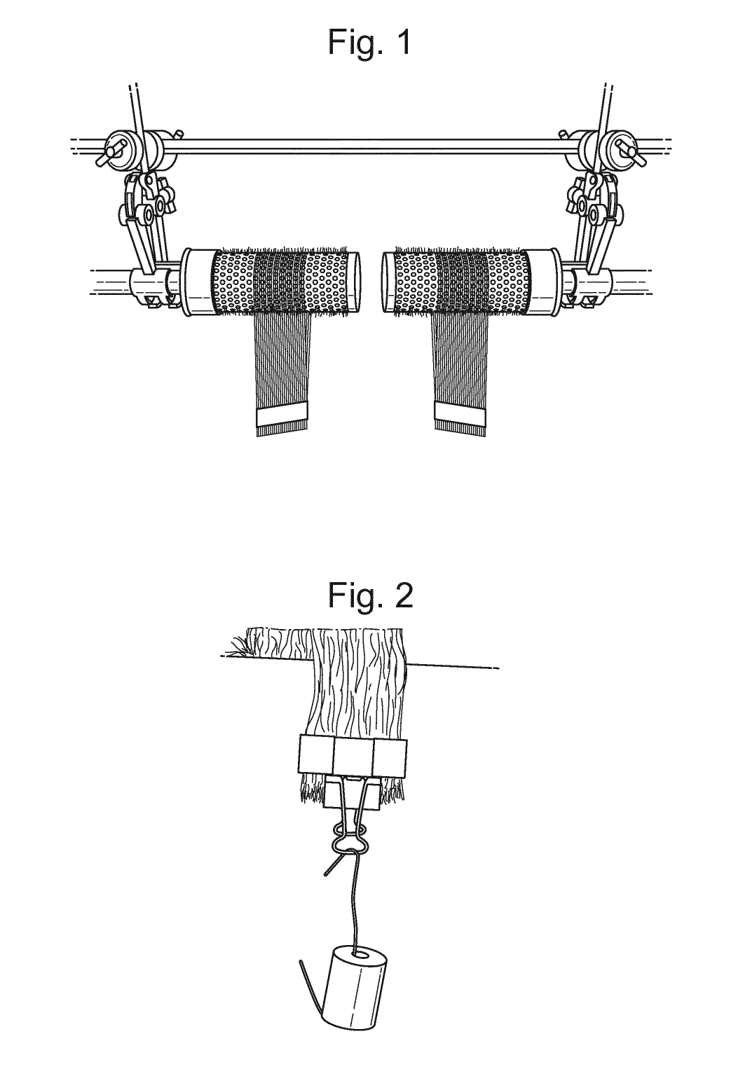

FIG. 1 is a photograph of hair switches positioned on hair brushes to measure draw mass, and

FIG. 2 is a photograph of a weight attached to a hair switch to measure draw mass.

We have surprisingly found that conditioning compositions comprising a conditioning gel phase can be stable in the presence of proteinaceous compounds or zwitterions if the gel structure is formed according to one of the processes described herein.

Standard processes for making conditioning gel phases result in compositions which are unstable in the presence of zwitterions or proteinaceous materials. This is because the proteinaceous material or zwitterion interferes with the formation of the surfactant structure.

Compositions of the invention have a surfactant structure which is resistant to disruption from such materials.

Preferably, the zwitterion is one or more amino acids. Where the zwitterion is more than one amino acid it is preferred that it is a protein, whether natural or synthetic or a derivative such as a glycoprotein, lipoporotein for example, or a polypeptide or oligopeptide.

Preferably, the composition comprises a proteinaceious material such as a protein, polypeptide, oligopeptide or short chain amino acid sequence.

Preferably, the proteinaceous material comprises hydrolysed protein such as hydrolysed keratin, hydrolysed wheat protein, hydrolysed collagen, hydrolysed gelatine, hydrolysed elastin or other hydrolysed component of the extracellular matrix, skin, bones or connective tissue whether extracted from animal or vegetable source.

Preferably, the zwitterions or proteinaceous material is present at from 0.001 to 10% wt. of the composition, more preferably from 0.1 to 8% wt.

Draw Mass is the mass required to draw a control hair switch through a comb or brush. Thus the more tangled the hair the greater the mass required to pull the switch through the comb or brush.

In a second aspect, the composition comprises a conditioning gel phase obtainable by:

forming a ‘comelt’ in a first vessel comprising fatty alcohol and cationic component and 0-15% wt. comelt of water (A);

adding the ‘comelt’ to a second vessel containing water at 50-60° C. (B); and

mixing, wherein the temperature of the mixture of the comelt and the water in the second vessel (B) is controlled such that it is maintained from 56-65° C., preferably from 58-62° C., more preferably 60° C., wherein the fatty alcohol has from 8 to 22 carbons and wherein the cationic component comprises from 0-70% wt. cationic component, cationic surfactants have the formula N⁺R¹R²R³R⁴, more preferably from 30-60% wt. cationic surfactant component, and wherein R¹, R², R³ and R⁴ are independently (C₁ to C₃₀) alkyl or benzyl and comprising a proteinaceoous material or zwitterion.

The comelting of the fatty alcohol and the cationic surfactant forms an isotropic phase. This means that the development of structure, i.e. the formation of the lamellar conditioning gel phase, can be controlled by the temperature and rate of mixing of the comelt and the water. The conditioning composition ultimately made using such conditioning gel phase has superior conditioning capability which is demonstrated by the reduced Detangling Draw.

The conditioning compositions made using a conditioning gel phase of the invention are superior products to those made mixing the water, fatty alcohol and cationic surfactant at around 70 C. Specifically, the superiority manifests itself in superior next day conditioning benefits where one would expect superior conditioning benefits to be due to increased deposition of solids thus leaving the hair lank and greasy the following day.

The improvement thus resides in the balance of thermal energy at the point of mixing the water with the comelt.

If the water is too cold then the comelt solidifies resulting in a poorly mixed system and this ultimately provides a composition of low viscosity. If the temperature of the water is too high then it is also too high at the point of mixing with the comelt and so forms vesicles. This also gives rise to lower viscosity in the conditioning composition formed with the resulting conditioning gel phase.

Preferably, the water in the second vessel is maintained at 56-60° C. and more preferably at 57-59° C.

Preferably, the comelt comprises from 45-90% wt. comelt fatty alcohol.

Preferably, the fatty alcohol comprises from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is particularly preferable.

The level of fatty alcohol in the conditioner of the invention (not just the conditioning gel phase) will generally range from 0.01 to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

Preferably, the comelt comprises from 10-40% wt. of the comelt cationic component.

In a most preferred embodiment the conditioning composition is made by first preparing a conditioning gel phase which is formed by adding cationic surfactants to fatty alcohol and stir at 85° C.

Gradually add this mixture to water, containing other ingredients, typically at 55° C., but at a temperature tailored to the composition to ensure mixture temperature is 60° C., this temperature maintained by external heating if required, and stir.

Cool this towards ambient by adding more water, and other ambient temperature ingredients, and use of external cooling if required, and stir.

Remaining components to the conditioning composition may then be added.

In an alternative embodiment the conditioning composition of the invention is obtainable by first forming a conditioning gel phase by:

forming a ‘comelt’ in a first vessel comprising fatty alcohol and cationic component and 0-15% wt. comelt water independently adding the ‘comelt’ and water to a mixing vessel mixing, wherein the temperature of the mixture of the ‘comelt’ and the water is maintained at from 56-65° C., preferably from 58-62° C., more preferably 60° C. when in the mixing vessel, wherein the fatty alcohol comprises from 8 to 22 carbons, wherein the cationic component comprises from 0-70% cationic component, cationic surfactants have the formula N⁺R¹R²R³R⁴, more preferably from 30-60% wt. cationic surfactant component, and wherein R¹, R², R³ and R⁴ are independently (C₁ to C₃₀) alkyl or benzyl and wherein the composition comprises a proteinaceoous material or zwitterion..

Conditioning compositions made using the conditioning gel phase of the invention are superior conditioning products. Specifically, they are thicker, despite having lower solids levels, and they are rinsed more easily. Products which are rinsed more easily use less water and so provide for a more sustainable future. These products are considered desirable by the environmentally aware consumer.

Preferably, the process is a continuous process.

The comelt of the invention forms an isotropic phase which means the development of structure, i.e. the formation of the lamellar conditioning gel phase, can be controlled. In this process the temperature of the mixture of comelt and water is controlled by modifying the temperature of water added to the mix. Water may be added in one go or it may be staged. Typically, a first water vessel is maintained at around 40° C. and is pumped into the mixing vessel while a second water vessel is maintained at a sufficient temperature to modify the temperature of the mixture of water with comelt such that it falls within the required range, i.e. from 56-65° C., preferably from 58-62° C., more preferably 60° C. in the mixing vessel.

The conditioning composition ultimately made using such conditioning gel phase exhibits improved conditioning characteristics which are not observed when the conditioning gel phase is formed in the comelt.

The improvement thus resides in the balance of thermal energy at the point of mixing the water with the comelt.

If too cold then one ends up with a poorly mixed system due to the tendency for the comelt to solidify and this ultimately provides a composition of low viscosity. If the temperature of the mix vesicles form. This also gives rise to lower viscosity in the conditioning composition formed in the long run.

Preferably, the comelt comprises from 45-90% wt. comelt fatty alcohol.

Preferably, the fatty alcohol comprises from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is particularly preferable.

The level of fatty alcohol in the conditioner of the invention (not just the conditioning gel phase) will generally range from 0.01 to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

Preferably, the comelt comprises from 10-40% wt. of the comelt cationic surfactant.

Inject this mixture into a flowing stream of water, containing other ingredients, the temperature of the water varied to ensure this mixture has a temperature of 60° C. and mix.

Cool this stream towards ambient by injection into a second water stream and mix.

In an alternative embodiment the composition comprises a conditioning gel phase obtainable by:

forming an aqueous isotropic solution of cationic component;

mixing the aqueous isotropic solution of cationic surfactant with molten fatty alcohol,

wherein the temperature during mixing the fatty alcohol with the isotropic cationic surfactant solution is maintained from 55° C. to 65° C. and wherein the fatty alcohol has from 8 to 22 carbons and wherein the composition comprises a proteinaceous material or zwitterion.

A conditioning composition made using a conditioning gel phase of the invention has been shown to be superior to compositions made by standard processes where the materials are mixed in water at around 70° C. The superior conditioning manifests itself in superior conditioner thickness (despite having lower solids levels) and next day clean feel and conditioning benefits. These are surprising since it would be expected that superior conditioning products usually leave the hair lank and greasy the following day sue to excessive deposition of solids.

Preferably, the temperature of the mixture of the aqueous isotropic solution and fatty alcohol is maintained at from 55° C. to 65° C.

Preferably, the molten fatty alcohol is added to the aqueous isotropic solution of cationic surfactant.

In this process the temperature of the mixture is controlled by modifying the temperature/rate of the mixture of the fatty alcohol and the cationic surfactant solution. The temperature needs to be carefully controlled in order to achieve the right conditioning gel phase structure. The improvement thus resides in the balance of thermal energy at the point of mixing the fatty alcohol with the isotropic mixture.

After formation of the gel phase further water and additional ingredients may be added in one go or it may be staged. Preferably the gel phase is cooled prior to addition of the water.

The conditioning composition ultimately made using such conditioning gel phase has improved conditioning capabilities.

Preferably, the temperature of the mixture of the fatty alcohol and aqueous isotropic solution is maintained at from 58° C. to 62° C.; most preferably at 60° C.

Preferably, and prior to addition to the isotropic mixture, the fatty alcohol is maintained at a temperature sufficient to maintain the fatty alcohol in a liquid phase. Preferably the fatty alcohol is maintained at from 80° C. to 85° C.

Preferably, the resulting conditioning gel phase is mixed with a mixer having a rotor tip speed of 10-34, preferably from 21-27 and especially preferably 24 ms-1.

Preferably, the fatty alcohol comprises from 8 to 22 carbon atoms, more preferably 16 to 22. Fatty alcohols are typically compounds containing straight chain alkyl groups. Examples of preferred fatty alcohols include cetyl alcohol, stearyl alcohol and mixtures thereof.

The level of fatty alcohol in the conditioner of the invention (not just the conditioning gel phase) will generally range from 0.01 to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

In an alternative embodiment the conditioning composition comprises a conditioning gel phase obtainable by forming an aqueous dispersion of fatty alcohol and amidoamine;

adding a cationic surfactant to the aqueous dispersion and mixing; and neutralising the amidoamine,

wherein the temperature of the mixture of cationic surfactant in the aqueous dispersion is maintained at from 56° C. to 67° C. and wherein the composition comprises a proteinaceoous material or zwitterion..

Conditioning compositions made with the conditioning gel phase of the invention have improved conditioning performance. More specifically, the conditioning compositions made using the conditioning gel phase of the invention are thicker, even when using a lower level of solids, and provide improved clean feel the following day. This is surprising since one usually associates improved conditioning with increased deposition of solids which results on greasiness and heaviness the next day. To provide the opposite is an unmet consumer need.

Preferably, the temperature of the aqueous dispersion is maintained above the melting temperature of the fatty alcohol, preferably at least 5° C. higher than the melting point of the fatty alcohol.

Preferably, the aqueous dispersion is formed by adding fatty alcohol to water heated and maintained at least the melting point of the fatty alcohol and preferably at least 5° C. above the melting point of the fatty alcohol. Preferably, the aqueous dispersion is maintained at a melting point sufficient to maintain the fatty alcohol in a liquid phase. Preferably, the temperature of the mixture of the aqueous dispersion is controlled such that it is maintained from 56-67° C., preferably from 58-65° C., more preferably 63° C.

Preferably, the temperature of the mixture of the aqueous dispersion and the cationic surfactant is maintained at from 56° C. to 67° C. More preferably, the temperature of the mix of the aqueous dispersion and the cationic surfactant is maintained at from 58° C. to 65° C.; most preferably at 63° C.

Controlling the temperature of the mixture of fatty alcohol and the cationic surfactant means controlling the formation of gel structure. In this process the temperature of the mixture of comelt and water is controlled by modifying the temperature/rate of the cationic surfactant to the fatty alcohol and an amidoamine surfactant aqueous mix. If too cold or too hot then a system having a mixture of structures results and this has poorer conditioning capability.

After formation of the gel phase further water and additional ingredients may be added in one go or it may be staged.

Preferably, the process is a batch process.

Preferably the mixing of the cationic surfactant with the aqueous dispersion is monitored by measurement of viscosity, such that when the viscosity change plateaus, the required degree association has occurred and then the amidoamine is neutralised. Typically, this mixing of the cationic surfactant and aqueous dispersion takes from 20 to 60 minutes.

The conditioning composition ultimately made using such conditioning gel phase has improved conditioning performance compared with an identical conditioning composition made with an identical formulation made using a standard process.

Preferably, the process comprises passing the contents of the mixture vessel through a mixer with rotor tip speed of 10-34, preferably from 21-27 and especially preferably 24 ms-1.

Preferably the aqueous dispersion comprises from 25 wt. % to 50 wt. %, more preferably from 35 to 45 wt. % of the total dispersion water.

Preferably the aqueous dispersion comprises from 4 to 20 wt. % of the total dispersion fatty alcohol.

Preferably the aqueous dispersion comprises from 1 to 5 wt. % of the total dispersion amidoamine.

Preferably the neutraliser added to the aqueous dispersion and cationic surfactant comprises sufficient neutraliser to neutralise at least 90 w t% of the cationic surfactant, more preferably at least 95% of the cationic surfactant, most preferably at least 99% of the cationic surfactant.

Preferably, the fatty alcohol comprises from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is particularly preferable.

The level of fatty alcohol in the conditioner of the invention (not just the conditioning gel phase) will generally range from 0.01 to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

Preferably, the conditioning gel phase is obtainable by adding a stearylamidopropyl dimethylamine and fatty alcohol to water at 60° C., maintain temperature by use of external heating, and stir.

Add a cationic surfactant, typically behentrimonium chloride, to this mixture, maintain temperature at 60° C. by use of external heating or cooling, and stir.

Add lactic acid to protonate stearylamidopropyl dimethylamine, maintain temperature at 60° C. by use of external heating or cooling, and stir.

Cool this towards ambient by adding more water, and other ambient temperature ingredients, and use of external cooling if required, and stir.

Further ingredients are then added to form a conditioning composition.

Suitable conditioning surfactants include those selected from cationic surfactants, used singly or in admixture. Preferably, the cationic surfactants have the formula N⁺R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethyleneoxy groups.

Suitable cationic surfactants for use in the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride,

hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN KDMP, ex Clariant.

Preferably, the cationic surfactant component of the comelt comprises from 0-70% cationic component, cationic surfactants have the formula N+R¹R²R³R⁴as described above, more preferably from 30-60% wt. cationic surfactant component.

Another example of a class of suitable cationic surfactants for use in the invention, either alone or together with one or more other cationic surfactants, is a combination of (i) and (ii) below:

(i) an amidoamine corresponding to the general formula (I):

R1CONH(CH2)mN(R2)R3 (I)

in which R¹ is a hydrocarbyl chain having 10 or more carbon atoms, R² and R³ are independently selected from hydrocarbyl chains of from 1 to 10 carbon atoms, and m is an integer from 1 to about 10; and

(ii) an acid.

As used herein, the term hydrocarbyl chain means an alkyl or alkenyl chain.

Preferred amidoamine compounds are those corresponding to formula (I) in which

R¹ is a hydrocarbyl residue having from about 11 to about 24 carbon atoms,

R² and R³ are each independently hydrocarbyl residues, preferably alkyl groups, having from 1 to about 4 carbon atoms, and m is an integer from 1 to about 4.

Preferably, R² and R³ are methyl or ethyl groups.

Preferably, m is 2 or 3, i.e. an ethylene or propylene group.

Preferred amidoamines useful herein include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof.

Particularly preferred amidoamines useful herein are stearamidopropyldimethylamine, stearamidoethyldiethylamine, and mixtures thereof.

Commercially available amidoamines useful herein include: stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

Acid may be any organic or mineral acid which is capable of protonating the amidoamine in the conditioner composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, lactic acid and mixtures thereof.

The primary role of the acid is to protonate the amidoamine in the hair treatment composition thus forming a tertiary amine salt (TAS) in situ in the hair treatment composition. The TAS in effect is a non-permanent quaternary ammonium or pseudo-quaternary ammonium cationic surfactant.

Suitably, the acid is included in a sufficient amount to protonate more than 95 mole% (293 K) of the amidoamine present.

Should an amidoamine of the type described herein be present then the corresponding acid component will not be present in the comelt. Instead it will be present in the water. Preferably, the water comprises protonating component at from 0.01 to 3% wt.

Accordingly, where the invention requires from 10-40% wt. comelt cationic surfactant, the cationic surfactant component may comprise amidoamine which is not protonated, i.e. it will not be cationic charged but will become protonated when added to the water and hence the protonating material contained therein.

Preferably, the cationic surfactant component of the comelt comprises from 0-70% cationic component, amidoamine corresponding to formula (I), more preferably from 30-60% wt. cationic surfactant component.

In conditioning compositions of the invention (not merely the conditioning gel phase), the level of cationic surfactant will generally range from 0.01% to 10%, more preferably 0.05% to 7.5%, most preferably 0.1% to 5% by weight of the composition.

Preferably, where a comelt is used, the comelt is maintained at a melting point sufficient to maintain the fatty alcohol in a liquid phase. Preferably, the comelt is maintained at from 80-85 C.

Preferably, the temperature of the mixture of the comelt and the water is controlled such that it is maintained from 56-65 C, prefer from 58-62 C, more preferably 60 C during mixing.

Preferably, the contents of the mixture vessel passed through a mixer with rotor tip speed of 10-34, preferably from 21-27 and especially preferably 24 ms-1.

In a further aspect there is provided a process for manufacturing a conditioning composition by forming a conditioning gel phase obtained as described above and then adding any remaining ingredients. Typical remaining ingredients include fragrances, silicones, fibre actives or other benefit agents.

Preferably, the conditioning composition is passed through a mixer with rotor tip speed of 10-34, preferably from 21-27 and especially preferably 24 ms-1 one more time after the remaining ingredients have been added.

Conditioning compositions of the invention or using conditioning gel phases of the invention also deposit silicone better than conventionally made conditioning compositions.

Accordingly, the compositions of the invention can contain, emulsified droplets of a silicone conditioning agent, for enhancing conditioning performance.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

The viscosity of the emulsified silicone itself (not the emulsion or the final hair conditioning composition) is typically at least 10,000 cst at 25oC the viscosity of the silicone itself is preferably at least 60,000 cst, most preferably at least 500,000 cst, ideally at least 1,000,000 cst. Preferably the viscosity does not exceed 109 cst for ease of formulation.

Emulsified silicones for use in the shampoo compositions of the invention will typically have an average silicone droplet size in the composition of less than 30, preferably less than 20, more preferably less than 10 micron, ideally from 0.01 to 1 micron. Silicone emulsions having an average silicone droplet size of 0.15 micron are generally termed microemulsions.

Emulsified silicones for use in the conditioner compositions of the invention will typically have an size in the composition of less than 30, preferably less than 20, more preferably less than 15. Preferably the average silicone droplet is greater than 0.5 micron, more preferably greater than 1 micron, ideally from 2 to 8 micron.

Silicone particle size may be measured by means of a laser light scattering technique, for example using a 2600D Particle Sizer from Malvern Instruments.

Examples of suitable pre-formed emulsions include Xiameter MEM 1785 and microemulsion DC2-1865 available from Dow Corning. These are emulsions/microemulsions of dimethiconol. Cross-linked silicone gums are also available in a pre-emulsified form, which is advantageous for ease of formulation.

A further preferred class of silicones for inclusion in shampoos and conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”.

Specific examples of amino functional silicones suitable for use in the invention are the aminosilicone oils DC2-8220, DC2-8166 and DC2-8566 (all ex Dow Corning).

Suitable quaternary silicone polymers are described in EP-A-0 530 974. A preferred quaternary silicone polymer is K3474, ex Goldschmidt.

Also suitable are emulsions of amino functional siliconoils with non ionic and/or cationic surfactant.

Pre-formed emulsions of amino functional silicone are also available from suppliers of silicone oils such as Dow Corning and General Electric. Specific examples include DC939 Cationic Emulsion and the non-ionic emulsions

DC2-7224, DC2-8467, DC2-8177 and DC2-8154 (all ex Dow Corning).

The total amount of silicone is preferably from 0.01 wt% to 10%wt of the total composition more preferably from 0.1 wt% to 5 wt%, most preferably 0.5 wt% to 3 wt% is a suitable level.

### EXAMPLE 1

5 g 10 inch (30 cm) Virgin (not chemically damaged) Indian hair switches [industry standard hair type ex. International Hair Importers and Products, Glendale, N.Y.] were base washed using 14% SLES, according to the base washing protocol.

### Base Washing Protocol

All switch washing to be done using the flow/temperature controlled taps. The flow rate is set at 4 litres/minute and a temperature of 35° C.-40° C.

1. Prep all of the syringes prior to starting to wash.
Pre-fill the syringe with the base wash and empty
Place the syringe on the balance and tare the balance
Fill the syringe to the required mark and check on the balance that the correct amount for the two washes has been weighed out
Repeat for each switch

2. Turn on tap and leave to stabilise for 30 seconds. The temperature and flow control is used by turning on the hot tap fully. Once the tap is turned on it is advisable to leave it running until all of the switches being treated in the session are done.

3. Wet out the switch by running it under the tap, remove excess water by running the first and middle finger down the length of the switch.

4. Lay the switch down flat on the edge of the sink and apply half of the measured Base Wash* (0.1 ml/g hair) evenly down the length of the switch.

5. Holding both ends of the switch. Gently massage the base wash into the hair for 30 seconds. Make sure to keep hold of both ends of the switch to avoid overly tangling the fibres.

6. Rinse for 30 seconds, running the fingers down the switch every 5 seconds. Remove excess water.

7. Apply the remainder of the Base Wash evenly down the length of the switch.

8. Gently massage the Base Wash into the hair for 30 seconds, again holding both ends of the switch to avoid excess tangling.

9. Rinse for 30 seconds, running the fingers down the switch every 10 seconds. Remove excess water.

10. Lay the switch down on the edge of the sink and using the WIDE teeth of a Matador Sawcut No 4 comb; carefully comb the tangles out of the switch. Comb down the switch from the root to the tip, starting at the tip and in sections work up slowly to the root. Once all the tangles have been combed out finish with the NARROW teeth of the comb.

11. Run the first and middle finger down the switch and either dry at 50° C. in the Drying Cabinet for a minimum of 2 hrs. Alternatively dry overnight at 20° C./50% Relative Humidity.

| | | | | Formulation |
|---|---|---|---|---|
| Ingredient | Active (%) | INCI Name | Material Name | (%) |
| Primary | 70 | Texapon N701 | SLES-1EO | 14.00 |
| Surfactant | | | | |
| Water | 100 | Aqua | Water | To 100% |

pH range 5.5-6.5

A 5 g 10″ hair switch has approx 7000 fibres.

The switches were then dried in 50° C. drying cabinet for two hours.

### Test Protocol

The switches were then washed with the standard shampoo control formulation (see Table 1). The wash consisted of massaging in 0.1 g shampoo per 1 g of hair, for 30 seconds, followed by a 30 second rinse (water flow rate 4 l/min), then repeating these two steps.

The switches were then tested for detangling benefit using various conditioner test formulations.

The conditioner was used at a concentration of 0.2 g of hair conditioner per 1 g of hair.

This was massaged into the switch for 1 minute and then rinsed for 5 seconds (water flow rate 4 l/min). The wet switch was placed onto a brush with a bulldog clip fastened to the glued end of the switch. The switch was placed on the brush such that from 5 cm to 20 cm was left hanging at the glued end. This situation is shown in FIG. 1, where two hair switches are positioned on two hair brushes, in preparation for the measurement of draw mass.

Weights were added to the glued end of the switch until the switch fell through the brush. The mass of the weight added to the switch when movement of the switch through the bristles of the brush is initiated is the draw mass. This situation is shown in FIG. 2, where a 50 g weight is attached to one end of a hair switch.

TABLE 1
Table 1 presents the shampoo control for assessing Detangling
Draw. The shampoo is made by standard processes.

| INCI Name (CTFA) | % (W/W) |
|---|---|
| Sodium Laureth Sulfate | 18.571 |
| Dimethiconol and Trideceth-10 and TEA- | 5.240 |
| Dodecylbenzenesulfonate | |
| Cocamidopropyl Betaine | 3.000 |
| Perfume | 0.750 |
| Ethylene Glycol Distearate/Sodium Laureth Sulphate/ | 9.302 |
| Cocomonoethanol amide | |
| Glycerin | 0.500 |
| Acrylates/Beheneth-25 Methacrylate Copolymer | 1.000 |
| Amodimethicone and cetrimonium chloride and trideceth-12 | 1.140 |
| Guar Hydroxypropyltrimonium Chloride | 0.225 |
| Mica and Titanium Dioxide | 0.150 |
| Acrylates/Styrene Copolymer | 0.500 |
| Gluconolactone | 0.100 |
| Trehalose | 0.100 |
| Adipic Acid | 0.100 |
| Sodium Sulfate | 0.100 |
| Disodium EDTA | 0.100 |
| Guar Hydroxypropyltrimonium Chloride | 0.075 |
| PEG-45M | 0.075 |
| Preservative | 0.100 |
| Helianthus Annuus (Sunflower) Seed Oil | 0.010 |
| Preservative | 0.060 |
| Sodium Hydroxide | 0.150 |
| Citric Acid Monohydrate | 0.15 |
| Water | up to 100 |

Conditioner Compositions:

The comparative formulation was made by standard processes. The inventive formulation was made by processes as described above.

| | Comparative | Inventive |
|---|---|---|
| INCI Name (CTFA) | % (W/W) | % (W/W) |
| Cetearyl Alcohol | 4.000 | 3.1500 |
| Dimethicone and | 4.290 | 4.2900 |
| Amodimethicone and PEG-7 | | |
| Propylheptyl Ether and | | |
| Cetrimonium Chloride | | |
| Behentrimonium Chloride | 1.630 | 1.3700 |
| Glycerin | 1.000 | 1.0000 |
| Perfume | 0.500 | 0.5000 |
| Stearamidopropyl | 0.375 | 0.3200 |
| Dimethylamine | | |
| Lactic Acid | 0.120 | 0.1000 |
| Disodium EDTA | 0.100 | 0.1000 |
| Preservative | 0.100 | 0.100 |
| Sunflower Seed Oil | 0.010 | 0.0100 |
| Arginine HCL | 0.010 | 0.0100 |
| Lysine HCl | 0.010 | 0.0100 |
| Preservative | 0.060 | 0.0600 |
| Dye | 0.00013 | 0.00013 |
| Dye | 0.00013 | 0.00013 |
| Ammonium Hydroxide | 0.02000 | 0.02000 |
| Chlorinated water | up to 100 | up to 100.00 |

1. A condition composition comprising from 0.4 to 8% wt. fatty alcohol having from 8-22 carbons, from 0.1 to 2% wt. cationic surfactant component, water, and wherein the composition confers a Draw Mass of from 1 to 250 g to hair treated with the conditioning composition and
wherein the Draw Mass is the mass required to draw a hair switch through a comb or brush as measured by first placing the hair switch onto the comb or brush, such that from 5 cm to 20 cm of hair is left hanging at the glued end of the switch, and then adding weights to the hanging end until the switch falls through the comb or brush, and wherein the conditioning composition includes a zwitterion or protenaceous material.

2. A composition comprising a conditioning gel phase obtained by:
forming a ‘comelt’ in a first vessel comprising fatty alcohol and cationic component and 0-15% wt. comelt of water (A);
adding the ‘comelt’ to a second vessel containing water at 50-60° C. (B); and
mixing,
wherein the temperature of the mixture of the comelt and the water in the second vessel (B) is controlled such that it is maintained from 56-65° C., wherein the fatty alcohol has from 8 to 22 carbons and wherein the cationic component comprises from 0-70% wt. cationic component, cationic surfactants have the formulaN±R¹R²R³R⁴, and wherein R¹, R², R³ and R⁴ are independently (C₁ to C₃₀) alkyl or benzyl, and wherein the composition includes a zwitterion or proteinaceous material.

3. A composition comprising a conditioning gel phase obtained by:
forming a ‘comelt’ in a first vessel comprising fatty alcohol and cationic component and 0-15% wt. comelt water
independently adding the ‘comelt’ and water to a mixing vessel,
mixing,
wherein the temperature of the mixture of the ‘comelt’ and the water is maintained at from 56-65° C., when in the mixing vessel, wherein the fatty alcohol comprises from 8 to 22 carbons, wherein the cationic component comprises from 0-70% cationic component, cationic surfactants have the formula N+R¹R²R³R⁴, and wherein R¹, R², R³ and R⁴ are independently (C₁ to C₃₀) alkyl or benzyl and wherein the composition includes a zwitterion or proteinaceous material.

4. A composition comprising a conditioning gel phase obtained by:
forming an aqueous isotropic solution of cationic surfactant;
mixing the aqueous isotropic solution of cationic surfactant with molten fatty alcohol
wherein the temperature during mixing the fatty alcohol with the isotropic cationic surfactant solution is maintained from 55° C. to 65° C. and wherein the fatty alcohol has from 8 to 22 carbons and and wherein the composition includes a zwitterion or proteinaceous material.

5. A composition comprising a conditioning gel phase obtained by:
forming an aqueous dispersion of fatty alcohol and amidoamine;
adding a cationic surfactant to the aqueous dispersion and mixing; and
neutralising the amidoamine,
wherein the temperature of the mixture of cationic surfactant in the aqueous dispersion is maintained at from 56° C. to 67° C. and wherein the composition include a zwitterion or proteinaceous material.
